(19) United States
(12) Patent Application Publication
(10) Pub. No.: US 2017/0172432 A1
(43) Pub. Date: Jun. 22, 2017

(54) Device for Measuring Psychological Signals

(71) Applicant: National Kaohsiung University of Applied Sciences, Kaohsiung (TW)

(72) Inventors: Jian-Chiun Liou, Kaohsiung (TW); Tien-Tsorng Shih, Kaohsiung (TW); Cheng-Hong Yang, Kaohsiung (TW)

(21) Appl. No.: 14/973,867

(22) Filed: Dec. 18, 2015

(51) Int. Cl. A61B 5/024; A61B 5/00

(52) U.S. Cl. CPC A61B 5/02416; A61B 5/0004; A61B 5/7278

(57) ABSTRACT

A device for measuring psychological signals includes a substrate, a light emitting unit, a photoelectric sensor and a microprocessor. The substrate includes a surface that is divided into an emission area and a reception area. The light emitting unit is arranged in the emission area and includes a plurality of light emitting sections. The plurality of light emitting sections irradiates a plurality of lights with different wavelengths on a predetermined location. The photoelectric sensor is arranged in the reception area, and is capable of detecting a plurality of reflected lights from the predetermined location and generating an electrical signal accordingly. The microprocessor is electrically connected to the photoelectric sensor, receives the electrical signal, and converts the electrical signal into a waveform signal. Thus, the detection accuracy can be improved. As such, the detection accuracy of the psychological condition of the user can be improved.

### BACKGROUND OF THE INVENTION

1. Field of the Invention

The present disclosure generally relates to a device for measuring psychological signals and, more particularly, to a device that measures psychological signals through photoelectric detection.

2. Description of the Related Art

Psychological signals can reflect the psychological condition of a user such that the health condition of the user can be monitored. For example, a heartbeat signal represents the beating frequency of the heart and can be used to determine the health condition of the user. The heartbeat signal can be measured through a cardiograph having electrode plates. However, it has been a common practice that the heartbeat signal is measured through photoelectric transmission.

A conventional photoelectric measuring device usually includes a light emitter and a photoelectric sensor. During the operation of the photoelectric measuring device, the light emitter may be placed on a skin surface of the user so that the light emitter can irradiate light on a blood vessel below the skin surface. In this regard, the light is reflected by the blood vessel. Then, the reflected light is received by the photoelectric sensor, which accordingly generates an electrical signal. The reflected light shows the information regarding the variation of the blood flow in the blood vessel. Since the blood flow is related to the heartbeat frequency, the electrical signal that is generated from the reflected light can also reflect the variation of the blood flow in the blood vessel. Thus, the electrical signal can be used as a heartbeat signal.

However, the light irradiated by the light emitter includes only one color, which means that the light emitter can only irradiate the light with a predetermined wavelength. However, when the light is reflected, the strength of the reflected light may be affected by some internal tissue in the human body. If the strength of the reflected light is too small, the photoelectric sensor will not be able to properly detect the reflected signal and cannot output the electrical signal that reliably reflects the variation of the blood flow. As a result, the detection accuracy is low.

In light of this, it is necessary to improve the conventional photoelectric measuring device.

### SUMMARY OF THE INVENTION

It is therefore the objective of this disclosure to provide a device for measuring psychological signals, which measures the psychological signals via different wavelengths of lights. As such, the detection accuracy is improved.

In an embodiment of the disclosure, a device for measuring psychological signals includes a substrate, a light emitting unit, a photoelectric sensor and a microprocessor. The substrate includes a surface that is divided into an emission area and a reception area. The light emitting unit is arranged in the emission area and includes a plurality of light emitting sections. Each of the plurality of light emitting sections includes at least one micro light-emitting diode. The plurality of light emitting sections is capable of irradiating a plurality of lights with different wavelengths on a predetermined location. The photoelectric sensor is arranged in the reception area. The photoelectric sensor is capable of detecting a plurality of reflected lights from the predetermined location and generating an electrical signal accordingly. The microprocessor is electrically connected to the photoelectric sensor and is capable of receiving the electrical signal and converting the electrical signal into a waveform signal. Thus, the detection accuracy can be improved.

In a form shown, the plurality of light emitting sections is arranged on the emission area in even intervals along a first direction. In this arrangement, the detection accuracy can be improved.

In the form shown, each of the plurality of light emitting sections has a same size. Each of the plurality of light emitting sections has a length and is spaced from an adjacent one of the plurality of light emitting sections at one of the even intervals along the first direction. The interval is not smaller than the length of the light emitting section. In this arrangement, the detection accuracy can be improved.

In the form shown, the length of the light emitting section is 20 μm. In this arrangement, the detection accuracy can be improved.

In the form shown, the plurality of light emitting sections includes a red light emitting section, a green light emitting section and a blue light emitting section. In this arrangement, the detection accuracy can be improved.

In the form shown, the at least one micro light-emitting diode of the red light emitting section includes a plurality of red light light-emitting diodes arranged in even intervals along a second direction perpendicular to the first direction. The at least one micro light-emitting diode of the green light emitting section includes a plurality of green light light-emitting diodes arranged in even intervals along the second direction, and the at least one micro light-emitting diode of the blue light emitting section includes a plurality of blue light light-emitting diodes arranged in even intervals along the second direction. In this arrangement, the detection accuracy can be improved.

In the form shown, the device for measuring psychological signals further includes a controller electrically connected to the light emitting unit and controlling at least two of the plurality of light emitting sections to irradiate at least two of the plurality of lights. In this arrangement, energy is saved while the detection accuracy is maintained.

In the form shown, the waveform signal is a heartbeat waveform. Thus, the beating condition of the heart can be conveniently determined.

In the form shown, the device for measuring psychological signals further includes an electronic device electrically connected to the microprocessor and receiving a waveform signal. The electronic device includes a display capable of displaying the waveform signal. Thus, the waveform signal can be received and observed by the user in a great convenience.

In the form shown, the microprocessor includes a wireless transmission module electrically connected to a wireless receiving module of the electronic device. Thus, convenient use is provided.

In the form shown, the electronic device is a mobile phone. Thus, convenient use is provided.

In a further embodiment of the disclosure, a device for measuring psychological signals includes a substrate, a light emitting unit, a photoelectric sensor, a microprocessor and an integrated circuit. The substrate includes a surface that is divided into an emission area and a reception area. The light emitting unit is arranged in the emission area and includes a plurality of light emitting sections. Each of the plurality of light emitting sections includes at least one micro light-emitting diode. The plurality of light emitting sections is capable of irradiating a plurality of lights with different wavelengths on a predetermined location. The photoelectric sensor is arranged in the reception area. The photoelectric sensor is capable of detecting a plurality of reflected lights from the predetermined location and generating an electrical signal accordingly. The microprocessor is electrically connected to the photoelectric sensor and is capable of receiving the electrical signal and converting the electrical signal into a waveform signal. Thus, the detection accuracy can be improved. The integrated circuit is electrically connected to the light emitting unit and is capable of controlling at least two of the plurality of light emitting sections to irradiate the plurality of lights. The integrated circuit is electrically connected to the photoelectric sensor to receive and convert the electrical signal into a waveform signal.

### BRIEF DESCRIPTION OF THE DRAWINGS

The present disclosure will become more fully understood from the detailed description given hereinafter and the accompanying drawings which are given by way of illustration only, and thus are not limitative of the present disclosure, and wherein:

FIG. 1 shows a structure of a device for measuring psychological signals according to an embodiment of the disclosure.

FIG. 2 still shows the structure of the device for measuring psychological signals according to the embodiment of the disclosure.

FIG. 3 shows another structure of the device for measuring psychological signals according to a modification of the embodiment of the disclosure.

FIG. 4 shows a connection between a microprocessor and an electronic device of the device for measuring psychological signals according to the embodiment of the disclosure.

FIG. 5 shows a use of the device for measuring psychological signals according to the embodiment of the disclosure.

In the various figures of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the disclosure.

### DETAILED DESCRIPTION OF THE INVENTION

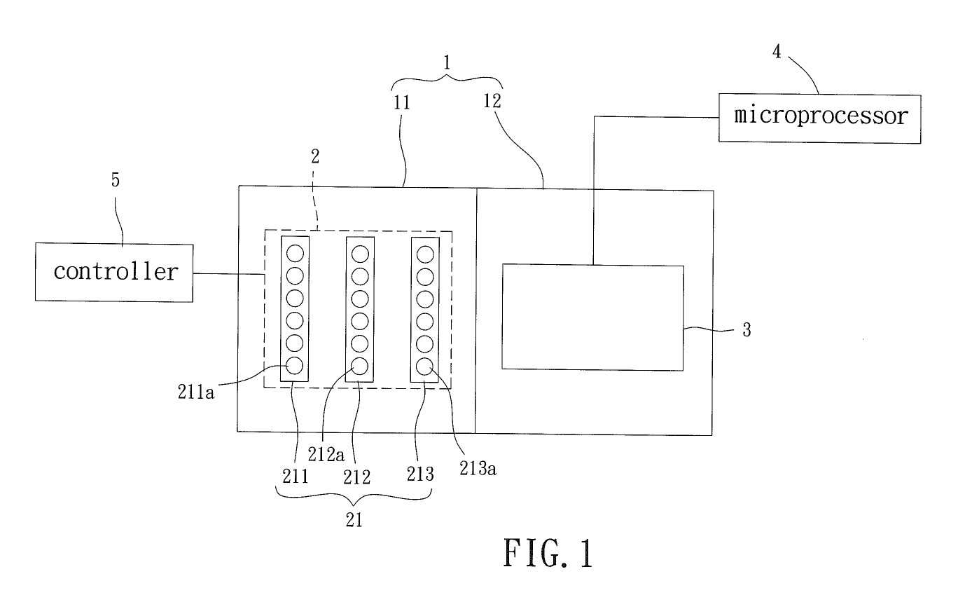

FIG. 1 shows a structure of a device for measuring psychological signals according to an embodiment of the disclosure. The device for measuring psychological signals includes a substrate 1, a light emitting unit 2, a photoelectric sensor 3 and a microprocessor 4. The light emitting unit 2 and the photoelectric sensor 3 are mounted on the substrate 1. The microprocessor 4 is electrically connected to the photoelectric sensor 3.

The substrate 1 includes a surface that is divided into an emission area 11 and a reception area 12. The light emitting unit 2 is arranged on the emission area 11, and the photoelectric sensor 3 is arranged on the reception area 12. The substrate 1 may include a flat or curved surface. For example, when the substrate 1 is a rigid plate, the substrate 1 includes a flat surface. When the substrate 1 is a flexible plate, the substrate 1 may include a flat or curved face.

The light emitting unit 2 includes a plurality of light emitting sections 21. Each of the plurality of light emitting sections 21 irradiates a light on a location. The lights of the plurality of light emitting sections 21 have different wavelengths.

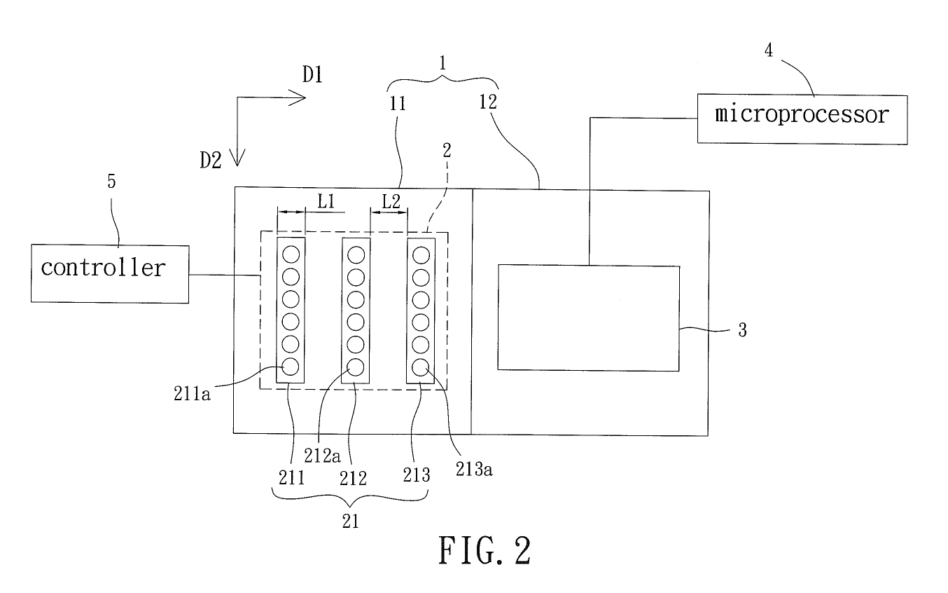

Referring to FIG. 2, the plurality of light emitting sections 21 is arranged on the emission area 11 in even intervals along a direction “D1.” If each of the plurality of light emitting sections 21 has the same size, each of the plurality of light emitting sections 21 has a length L1 and is spaced from the adjacent emitting section 21 at an interval L2 along the direction “D1”. The interval L2 is not smaller than the length L1 of the light emitting section 21. As such, the irradiated lights of the plurality of light emitting sections 21 will not interfere with each other through the arrangement of the intervals L2. The detection accuracy is improved. Besides, based on the design that the interval L2 is not smaller than the length L1, the irradiated lights of the plurality of light emitting sections 21 can be prevented from interfering with each other without having to excessively increase the mounting area of the light emitting unit 2, thus improving the detection accuracy.

Referring to FIGS. 1 and 2, each of the plurality of light emitting sections 21 preferably irradiates the light with a different wavelength. The quantity of the plurality of light emitting sections 21 is not limited. In the embodiment, there are three light emitting sections 21, including a red light emitting section 211, a green light emitting section 212 and a blue light emitting section 213. The red light emitting section 211 is used to irradiate the red light, the green light emitting section 212 is used to irradiate the green light, and the blue light emitting section 213 is used to irradiate the blue light. Moreover, each of the plurality of light emitting sections 21 can also emit the light with mixed colors. For example, one of the plurality of light emitting sections 21 can emit a mixture of red light and green light, green light and blue light, or red light and blue light. Furthermore, each of the plurality of light emitting sections 21 can also emit infrared light (IR light). As such, the light emitting unit 2 is able to irradiate different kinds of lights with different wavelengths. When the light emitting unit 2 irradiates the lights on a detected part inside the body, although some part of the lights may contain the wavelength that cannot penetrate certain tissue in the body, another part of the lights is still able to reach the detected part. Thus, the light can be reflected and used to detect the psychological condition of the patient. The detection accuracy is high.

Each of the plurality of light emitting sections 21 contains at least one micro light-emitting diode (μ LED). The size of the micro LED is 20 μm by 20 μm. Therefore, arrangement of the micro LED can not only reduce the volume and power consumption of the light emitting unit 2, but also efficiently irradiate the detected part with the light having a finer scale (through the use of micro LEDs). Thus, the detection accuracy is high.

In the embodiment, based on the arrangement of the red light emitting section 211, the green light emitting section 212 and the blue light emitting section 213, the red light emitting section 211 includes a plurality of red light LEDs 211a arranged in even intervals along a direction D2, the green light emitting section 212 includes a plurality of green light LEDs 212a arranged in even intervals along the direction D2, and the blue light emitting section 213 includes a plurality of red light LEDs 213a arranged in even intervals along the direction D2. The direction D2 is perpendicular to the direction D1. In the arrangement, the plurality of light emitting sections 21 includes a plurality of micro LEDs arranged in the form of an array. As such, when the plurality of light emitting sections 21 irradiates the lights with different wavelengths on a detected part in the body, it can be ensured that at least some part of the lights is able to reach the detected part due to its wavelength. As a result, the light can be reflected and used to detect the psychological information, improving the detection accuracy.

In the above structure, if each of the plurality of light emitting sections 21 contains only one micro LED along the direction D1, the length L1 of the light emitting section 21 is 20 μm. Accordingly, each of the plurality of light emitting sections 21 is able to properly irradiate the light on the predetermined location, thus improving the detection accuracy.

The photoelectric sensor 3 is mounted on the reception area 12 and can receive a plurality of reflected lights from the detected part. As such, the photoelectric sensor 3 can generate an electrical signal according to the plurality of reflected lights. The photoelectric sensor 3 may be any sensor capable of receiving lights and generating the electrical signal accordingly.

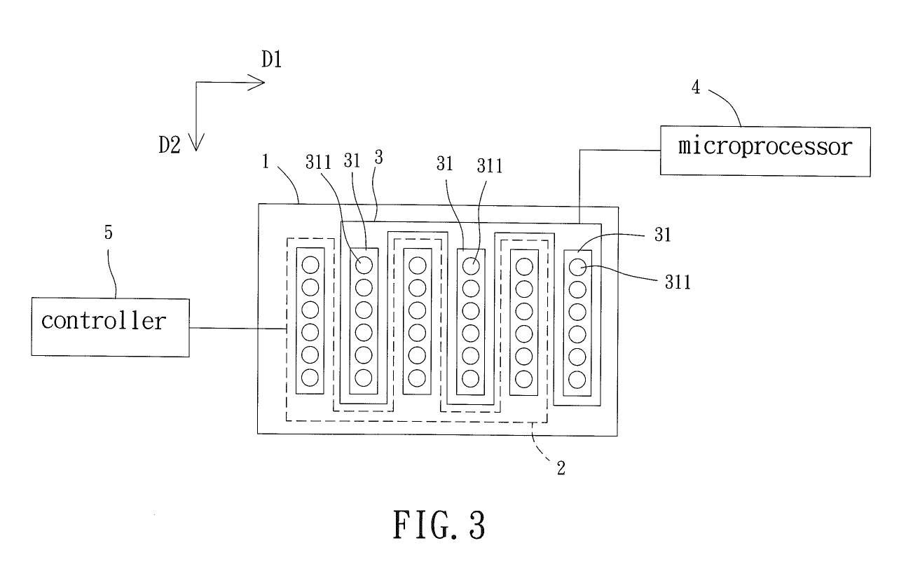

Referring to FIGS. 1 and 2 again, the light emitting unit 2 and the photoelectric sensor 3 may be arranged on different faces of the substrate 1. Alternatively, referring to FIG. 3, the photoelectric sensor 3 may also include a plurality of sensing sections 31 interlaced with the plurality of light emitting sections 21 along the direction “D1.” In this regard, each of the plurality of sensing sections 31 includes a plurality of sensing units 311 arranged in even intervals along the direction D2. The plurality of sensing units 311 is able to receive one or more reflected lights and to accordingly generate the electrical signal.

The microprocessor 4 is electrically connected to the photoelectric sensor 3 in order to receive the electrical signal, and then converts the electrical signal into a waveform signal. The microprocessor 4 may be any processor with a logic calculation function and a statistical analysis function. The microprocessor 4 is able to execute a signal processing procedure, which can convert the electrical signal into a waveform signal associated with the psychological condition, as it can be readily appreciated by the persons having ordinary skill in the art.

Referring to FIGS. 1 and 2 again, the device for measuring psychological signals according to the embodiment of the disclosure may further include a controller 5 electrically connected to the light emitting unit 2. The controller 5 is able to control at least two of the plurality of light emitting sections 21 to irradiate the lights. For example, based on the arrangement of the red light emitting section 211, the green light emitting section 212 and the blue light emitting section 213, the controller 5 is able to control the red light emitting section 211 and the green light emitting section 212, the red light emitting section 211 and the blue light emitting section 213, or the green light emitting section 212 and the blue light emitting section 213 to irradiate the lights. Thus, arrangement of the controller 5 is able to maintain the detection accuracy of the psychological signals without having to consume a large amount of power. Advantageously, energy is saved and high detection accuracy is provided.

Moreover, the microprocessor 4 and the controller 5 may also be integrated as an integrated circuit formed on a semiconductor substrate. The integrated circuit may be electrically connected to the light emitting unit 2 and the photoelectric sensor 3. The detailed operations thereof have been described above,

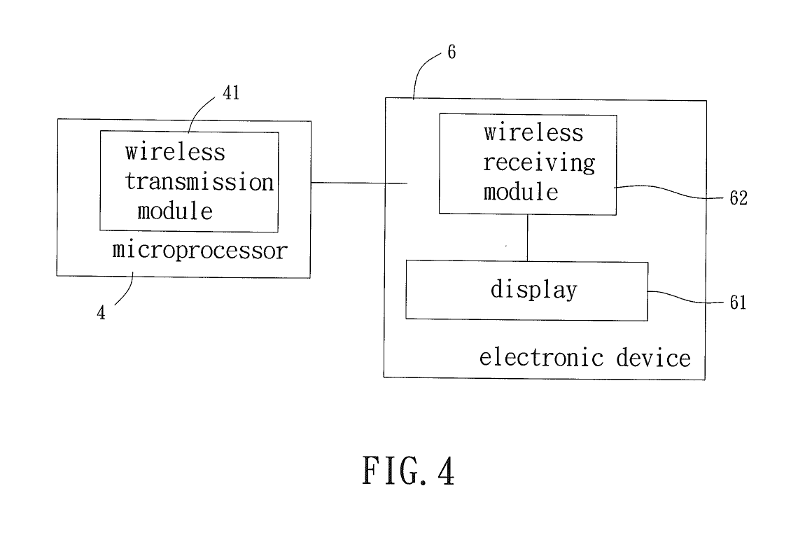

Referring to FIG. 4, the device for measuring psychological signals according to the embodiment of the disclosure may further include an electronic device 6 which is electrically connected to the microprocessor 4 in order to receive the waveform signal. The electronic device 6 includes a display 61 capable of displaying the waveform signal. The electronic device 6 may be a portable electronic device, such as a mobile phone or a tablet PC. When the waveform signal is a heartbeat waveform, the display 61 may display the heartbeat waveform. The beating condition of the heart can be conveniently observed through the heartbeat waveform. In addition, through the electronic device 6, the psychological signals can be received and observed by the user in a great convenience. Moreover, the electronic device 6 can be electrically connected to the microprocessor 4 in a wired manner, or in a wireless manner in which the microprocessor 4 includes a wireless transmission module 41 electrically connected to a wireless receiving module 62 of the electronic device 6. The wireless transmission module 41 and the wireless receiving module 62 can be a conventional wireless transmission configuration such as Bluetooth, thereby providing a convenient use.

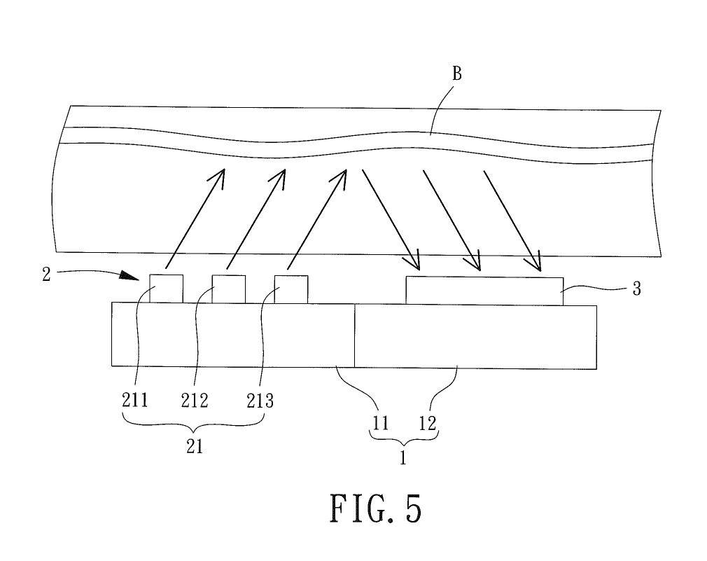

Referring to FIG. 5, when the device for measuring psychological signals according to the embodiment of the disclosure is used to measure the heartbeat rate of the user, the substrate 1 may be placed on a skin surface of the user such that the light emitting unit 2 and the photoelectric sensor 3 may face the skin of the user. In this regard, the skin surface contains a blood vessel “B” serving as the detected part, and the plurality of light emitting sections 21 irradiates the detected part with the lights having different wavelengths. The lights must penetrate the skin tissue in order to reach the blood vessel “B.” In this regard, due to the diversity in the wavelength of the lights, although some part of the lights may contain the wavelength that cannot reach the blood vessel “B” as it is obstructed by the internal tissue, another part of the lights is still able to reach the blood vessel “B.”

When the lights are irradiated on the blood vessel “B,” the hemoglobin in the blood vessel “B” will absorb the lights, thereby reflecting the lights. When the blood flow of the blood vessel “B” changes as the heart beats (i.e. the concentration of the hemoglobin changes), the reflected lights also change. As such, the electrical signal generated from the reflected lights can represent the beating condition of the heart. In this regard, the microprocessor 4 can receive and convert the electrical signal into a waveform signal indicative of the beating condition of the heart, thereby obtaining the required psychological signals.

In summary, the device for measuring psychological signals according to the embodiment of the disclosure is able to emit different wavelengths of lights when detecting the psychological signals of the patient. Due to the diversity in wavelength of the lights, the psychological signals of the patient can still be detected even if some part of the lights contains the wavelength that is obstructed by certain body tissue. Thus, the detection accuracy is high.

Although the disclosure has been described in detail with reference to its presently preferable embodiments, it will be understood by one of ordinary skill in the art that various modifications can be made without departing from the spirit and the scope of the disclosure, as set forth in the appended claims.

What is claimed is:

1. A device for measuring psychological signals, comprising:
a substrate comprising a surface, wherein the surface is divided into an emission area and a reception area;
a light emitting unit arranged in the emission area and comprising a plurality of light emitting sections, wherein each of the plurality of light emitting sections comprises at least one micro light-emitting diode, wherein the plurality of light emitting sections is capable of irradiating a plurality of lights with different wavelengths on a predetermined location;
a photoelectric sensor arranged in the reception area, wherein the photoelectric sensor is capable of detecting a plurality of reflected lights from the predetermined location and generating an electrical signal accordingly; and
a microprocessor electrically connected to the photoelectric sensor and capable of receiving the electrical signal and converting the electrical signal into a waveform signal.

2. The device for measuring psychological signals as claimed in claim 1, wherein the plurality of light emitting sections is arranged on the emission area in even intervals along a first direction.

3. The device for measuring psychological signals as claimed in claim 2, wherein each of the plurality of light emitting sections has a same size, wherein each of the plurality of light emitting sections has a length and is spaced from an adjacent one of the plurality of light emitting sections at one of the even intervals along the first direction, and wherein the interval is not smaller than the length of the light emitting section.

4. The device for measuring psychological signals as claimed in claim 3, wherein the length of the light emitting section is 20 μm.

5. The device for measuring psychological signals as claimed in claim 3, wherein the plurality of light emitting sections comprises a red light emitting section, a green light emitting section and a blue light emitting section.

6. The device for measuring psychological signals as claimed in claim 5, wherein the at least one micro light-emitting diode of the red light emitting section comprises a plurality of red light light-emitting diodes arranged in even intervals along a second direction perpendicular to the first direction, wherein the at least one micro light-emitting diode of the green light emitting section comprises a plurality of green light light-emitting diodes arranged in even intervals along the second direction, and wherein the at least one micro light-emitting diode of the blue light emitting section comprises a plurality of blue light light-emitting diodes arranged in even intervals along the second direction.

7. The device for measuring psychological signals as claimed in claim 1, further comprising a controller electrically connected to the light emitting unit and controlling at least two of the plurality of light emitting sections to irradiate at least two of the plurality of lights.

8. The device for measuring psychological signals as claimed in claim 1, wherein the waveform signal is a heartbeat waveform.

9. The device for measuring psychological signals as claimed in claim 1, further comprising an electronic device electrically connected to the microprocessor and receiving a waveform signal, wherein the electronic device comprises a display capable of displaying the waveform signal.

10. The device for measuring psychological signals as claimed in claim 9, wherein the microprocessor comprises a wireless transmission module electrically connected to a wireless receiving module of the electronic device.

11. The device for measuring psychological signals as claimed in claim 9, wherein the electronic device is a mobile phone.

12. A device for measuring psychological signals, comprising:
a substrate comprising a surface, wherein the surface is divided into an emission area and a reception area;
a light emitting unit arranged in the emission area and comprising a plurality of light emitting sections, wherein each of the plurality of light emitting sections comprises at least one micro light-emitting diode, wherein the plurality of light emitting sections is capable of irradiating a plurality of lights with different wavelengths on a predetermined location;
a photoelectric sensor arranged in the reception area, wherein the photoelectric sensor is capable of detecting a plurality of reflected lights from the predetermined location and generating an electrical signal accordingly;
a microprocessor electrically connected to the photoelectric sensor and capable of receiving the electrical signal and converting the electrical signal into a waveform signal; and
an integrated circuit electrically connected to the light emitting unit and capable of controlling at least two of the plurality of light emitting sections to irradiate the plurality of lights, wherein the integrated circuit is electrically connected to the photoelectric sensor to receive and convert the electrical signal into a waveform signal.

13. The device for measuring psychological signals as claimed in claim 12, wherein the plurality of light emitting sections is arranged on the emission area in even intervals along a first direction.

14. The device for measuring psychological signals as claimed in claim 13, wherein each of the plurality of light emitting sections has a same size, wherein each of the plurality of light emitting sections has a length and is spaced from an adjacent one of the plurality of light emitting sections at one of the even intervals along the first direction, and wherein the interval is not smaller than the length of the light emitting section.

15. The device for measuring psychological signals as claimed in claim 14, wherein the length of the light emitting section is 20 μm.

16. The device for measuring psychological signals as claimed in claim 14, wherein the plurality of light emitting sections comprises a red light emitting section, a green light emitting section and a blue light emitting section.

17. The device for measuring psychological signals as claimed in claim 16, wherein the at least one micro light-emitting diode of the red light emitting section comprises a plurality of red light light-emitting diodes arranged in even intervals along a second direction perpendicular to the first direction, wherein the at least one micro light-emitting diode of the green light emitting section comprises a plurality of green light light-emitting diodes arranged in even intervals along the second direction, and wherein the at least one micro light-emitting diode of the blue light emitting section comprises a plurality of blue light light-emitting diodes arranged in even intervals along the second direction.

18. The device for measuring psychological signals as claimed in claim 12, wherein the waveform signal is a heartbeat waveform.
